(19) United States
(12) Patent Application Publication
(10) Pub. No.: US 2014/0052165 A1
(43) Pub. Date: Feb. 20, 2014

(54) INTERCHANGEABLE SURGICAL SCALPEL

(71) Applicant: Edward BILENSKI, Butler, NJ (US)

(72) Inventor: Edward BILENSKI, Butler, NJ (US)

(21) Appl. No.: 13/967,966

(22) Filed: Aug. 15, 2013

Related U.S. Application Data
(60) Provisional application No. 61/684,317, filed on Aug. 17, 2012.

(51) Int. Cl. A61B 17/3211

(52) U.S. Cl. CPC A61B 17/3211
USPC 606/170

(57) ABSTRACT

A cutting knife allows multiple different blade tips to be removably attached to a handle. The handle may be a pen-type handle that uses an activation button to move a spring-loaded plunger between an extended and a retracted position, typically using a cam and cam follower. The interchangeable blade tips may use spring-loaded cutting blades that retract, and preferably lock, within a sheath when a plunger is not pushing the blade into an extended position. Thus, the cutting blade is safely stored within the sheath when the plunger is retracted, as well as when the interchangeable blade tip is detached from the handle. The interchangeable blade tips may be disposable or reusable. The cutting knife may be configured as a surgical scalpel in which the cutting blade is a scalpel blade. In such applications, the pen-type handle may be of autoclavable materials so that it may be reusable after sterilization.

### RELATED APPLICATIONS

This application claims the benefit of priority to U.S. Provisional Application No. 61/684,317, filed Aug. 17, 2012, the entire contents of which are hereby incorporated by reference.

### BACKGROUND

A typical surgical scalpel includes a handle portion and a blade portion. The cutting blades are very sharp and often pointed, so care must be taken to prevent accidental cuts when the scalpel is being handled, both prior to and after use. Additionally, it is generally desirable to dispose of contaminated surgical instruments after a single use. Disposal of used scalpels can pose a hazard because their shape blades can puncture trash bags and body fluids on the blades must be treated as a biohazard.

### SUMMARY

The various embodiments provide devices and methods for an interchangeable cutting knife, such as a surgical scalpel, that allows safe handling and disposal of used and/or contaminated portions.

Various embodiments of an interchangeable cutting knife, such as surgical scalpel overcome limitations of existing cutting knives, including surgical scalpels, by using a pen-type activation mechanism in conjunction with an interchangeable blade tip that locks the cutting blade within a sheath during assembly, storage (e.g., while on a surgical tray), handling, and/or disassembly for disposal. The separate handle and interchangeable blade tip allow for the disposal of the interchangeable blade tip with the blade retracted and secure, as well as reuse of the handle (e.g., after sterilization in an autoclave in the case of surgical scalpels).

### BRIEF DESCRIPTION OF THE DRAWINGS

The accompanying drawings, which are incorporated herein and constitute part of this specification, illustrate embodiments of the invention, and, together with the general description given above and the detailed description given below, serve to explain features of the invention.

FIG. 1A is an isomeric view of a cutting knife assembly in a retracted position in accordance with an embodiment.

FIG. 1B is an isomeric view of a cutting knife assembly in an extended position in accordance with an embodiment.

FIG. 1C is an isomeric view of a cutting knife assembly in accordance with an embodiment in a disassembled state with the cutting knife assembly exposed.

FIG. 1D is an isomeric view of a cutting knife assembly in accordance with an embodiment in a disassembled state.

FIG. 1E is an isomeric view of a pen-type handle in accordance with an embodiment.

FIG. 1F is an isomeric view of a sheath in accordance with an embodiment.

FIG. 1G is an isomeric view of a cutting blade in accordance with an embodiment.

FIG. 1H is an isomeric view of an interchangeable blade tip in accordance with an embodiment.

FIG. 1J is an isomeric view of an extended cutting blade in accordance with an embodiment.

FIG. 2A is a sectional view of a disassembled interchangeable blade tip and handle in accordance with an embodiment.

FIG. 2B is a sectional view of an assembled interchangeable blade tip and handle prior to button engagement in accordance with an embodiment.

FIG. 2C is a sectional view of an assembled interchangeable blade tip and handle during initial button engagement when the lock mechanism is being disengaged in accordance with an embodiment.

FIG. 2D is a sectional view of an assembled interchangeable blade tip and handle in an extended position after button engagement in accordance with an embodiment.

FIG. 3 is a cutaway/exploded view of interchangeable blade tip components in accordance with an embodiment.

FIG. 4 is an isomeric view of an interchangeable cutting knife and a plurality of interchangeable blade tips in accordance with an embodiment.

FIG. 5A is a sectional view of a cutting knife assembly in a retracted position in accordance with an embodiment having multiple, selectable blade tips.

FIG. 5B is a sectional view of a cutting knife assembly in an extended position in accordance with an embodiment having multiple, selectable blade tips.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The word “exemplary” is used herein to mean “serving as an example, instance, or illustration.” Any implementation described herein as “exemplary” is not necessarily to be construed as preferred or advantageous over other implementations.

The words “surgical scalpel,” “interchangeable surgical scalpel,” and “surgical scalpel assembly” as applied to the description of embodiments are used interchangeably to refer to an example cutting knife assembly. The words “handle,” “spring-loaded handle,” “pen-type handle,” and “handle assembly” as applied to the description of embodiments are used interchangeably. The words “interchangeable blade tip” and “blade tip assembly” as applied to the description of embodiments are also used interchangeably.

Embodiments are described herein with reference to a cutting blade or scalpel. However, a number of different knife or cutting implements may be used with and in the embodiments and are contemplated in the future, all of which may benefit from the various embodiments. Example surgical cutting implements include, e.g., incision scalpels, stab scalpels, crescent scalpels, round scalpels, keratome/slit scalpels, paracentesis scalpels, MVR scalpels, implant scalpels, side port scalpels, and the like. Example non-surgical cutting implements include trimming tools, carpet cutting tools, woodworking tools, dissection tools, and fabric cutters. The embodiments may also be used with sharp points, such as may be used to make holes in fabrics, animal hides, plastics, and the like. For ease of references, the term cutting blade is used generically to refer to any of a variety of knife, cutting blades and sharp points, and it should be understood that references to terminology and/or technical details related to a cutting blade are for illustrative purposes only, and are not intended to limit the scope of the claims to a particular type of cutting implement or technology unless specifically recited in the claim language.

Existing cutting blades are either fixed to handles (and can employ a guard when not in use) or retract into the handle in a manner similar to a utility knife. The majority of known surgical scalpels can result in exposure to the blade during assembly, storage (i.e., while on a surgical tray), handling, and/or disassembly by surgical personnel. For cutting blades that retract in the manner of a utility knife, the operation requires that the user maintain force for a sufficient time to fully retract the blade, and a user may believe the blade to be fully retracted when, in fact, the blade is still partially exposed. Additionally, since many retractable scalpels use activation mechanisms in different locations, operating room personnel will often position the blade in its exposed use position on the surgical tray, and thus expose the blade when passing the scalpel to the operating physician. Thus, it would be beneficial to have a surgical scalpel that minimizes exposure of the blade during assembly, storage (i.e., while on a surgical tray), handling, and/or disassembly by surgical personnel, yet is as easy to operate as a retractable pen.

The various embodiments of an interchangeable cutting knife overcome limitations of existing scalpels by using a pen-type activation mechanism in conjunction with an interchangeable blade tip that locks the cutting blade within a sheath during assembly, storage (i.e., while on a surgical tray), handling, and/or disassembly for disposal by surgical personnel. The separate handle and interchangeable blade tip allow for the disposal of the interchangeable blade tip with the blade retracted and secure, as well as reuse of the handle if desired (e.g., after sterilization in an autoclave). In certain embodiments, the activation mechanism of the handle and the blade tip can be configured for selected activation and retraction of multiple blade tips. The embodiments of an interchangeable cutting knife may also meet critical OSHA requirements. The safety device cannot be reused after a procedure is completed and surgical instruments are disposed (because the disposed tip requires the plunger to unlock. One-hand activation allows users to extend and retract the blade with one hand during the procedure due to the easy pen-style push button actuator structure. The one-hand operation allows users to keep their hands behind the blade during procedures. The enclosed sheath of the disposable tip holds a contaminated blade after use to prevent spread of contamination from body fluids and hazards posed by an exposed blade.

A surgical scalpel in accordance with an embodiment may include a pen-type handle and an interchangeable blade tip. The pen-type handle has an activation button at a proximal end. The pen-type handle may be configured so that depressing the button moves a spring-loaded plunger between retracted and extended positions at a distal end of the pen-type handle. The interchangeable blade tip has a sheath dimensioned to be removably attached at a proximal end to the distal end of the pen-type handle. The interchangeable blade tip further may include a spring-loaded cutting blade configured to engage the spring-loaded plunger and extend to an exposed use position when the spring-loaded plunger is in the extended position. The cutting blade may be any form of sharp point or knife blade, including a surgical scalpel blade. The spring-loaded cutting blade of the interchangeable blade tip may further be configured to disengage the spring-loaded plunger and retract to a safe position when the spring-loaded plunger is in the retracted position.

An embodiment of the cutting knife may include a lock mechanism configured to secure the cutting blade within the sheath. The spring-loaded cutting blade may further include a resilient member that engages a portion of the sheath when in a retracted position so as to lock the cutting blade within the sheath. The spring-loaded plunger may then unlock the cutting blade from the sheath by using a member that disengages the resilient member of the cutting blade from the sheath during initial movement into an extended position. In one embodiment, the resilient member may include a tapered catch at an end of a leaf spring. To assist in the operation of the locking mechanism, the sheath and pen-type handle may be dimensioned to align the resilient member of the cutting blade with the member that disengages on the plunger.

In an embodiment the interchangeable blade tip may further include a grip surface on a proximal portion of the sheath, and the interchangeable blade tip may further include a channel on an inner portion of the sheath to guide the cutting blade, and the interchangeable blade tip may further include a bar spring between an inner portion of the sheath and the cutting blade.

In another embodiment, the cutting knife may include a handle and an interchangeable blade tip. The handle has a hollow barrel with a proximal opening in a proximal end and a distal opening in a distal end. A button may be used for activation and has a proximal end, a distal end, and a flange, wherein the button is dimensioned to extend its proximal end through the proximal opening of the hollow barrel and be retained by the flange. A cam follower may be positioned adjacent the distal end of the button and configured to engage a cam on an inner surface of the hollow barrel to move between an extended position and a retracted position. A plunger may be located within the hollow barrel and have a proximal end, a distal end, and spring stop. The proximal end of the plunger may be dimensioned to engage a distal end of the cam follower, such that activation of the button moves the plunger through the four step-down positions as found in operation of a pen. A plunger spring may be configured to engage the spring stop on the plunger and the hollow barrel so as to retract the plunger from an engagement position when the cam follower is in the retracted position. The interchangeable blade tip may include a hollow sheath having proximal opening in a proximal end, a distal opening in a distal end, blade guides on an inner surface, and a blade stop at a distal end of the blade guides. The proximal end may be dimensioned and configured for removable attachment to the distal opening of the hollow barrel. A cutting blade may be located within the sheath and has a cutting surface at a distal end, a longitudinal edge for engaging the blade guides, a spring stop, and a proximal end dimensioned to engage a distal end of the plunger when the plunger is in the engagement position. A blade spring may be configured to engage the spring stop on the cutting blade and the hollow sheath so as retract the cutting blade within the sheath when the plunger is disengaged from the proximal end of the cutting blade.

Optionally, this embodiment of the cutting knife also may include a lock mechanism for the cutting blade to secure it within the sheath. The cutting blade may further include a resilient member that engages a portion of the sheath when in a retracted position so as to lock the cutting blade within the sheath. The plunger may then unlock the cutting blade from the sheath by using a member that disengages the resilient member of the cutting blade from the sheath during initial movement into an extended position. In one embodiment, the resilient member may include a tapered catch at an end of a leaf spring. To assist in the operation of the locking mechanism, the sheath and hollow barrel may be dimensioned to align the resilient member of the cutting blade with the member that disengages on the plunger.

In a further embodiment of the surgical scalpel, the blade tip may include a plurality of spring-mounted cutting blades. A pen-type mechanism in the handle of the scalpel assembly can include blade-selection actuators so that a selected one of the plurality of cutting blades will engage a plunger for movement into the extended and retracted positions.

In these and additional embodiments, the handle may be made from autoclavable materials, the interchangeable blade tip may be made from autoclavable materials, the interchangeable blade tip may be made from disposable materials, and the interchangeable blade tip may further include a grip surface on a proximal portion of the sheath.

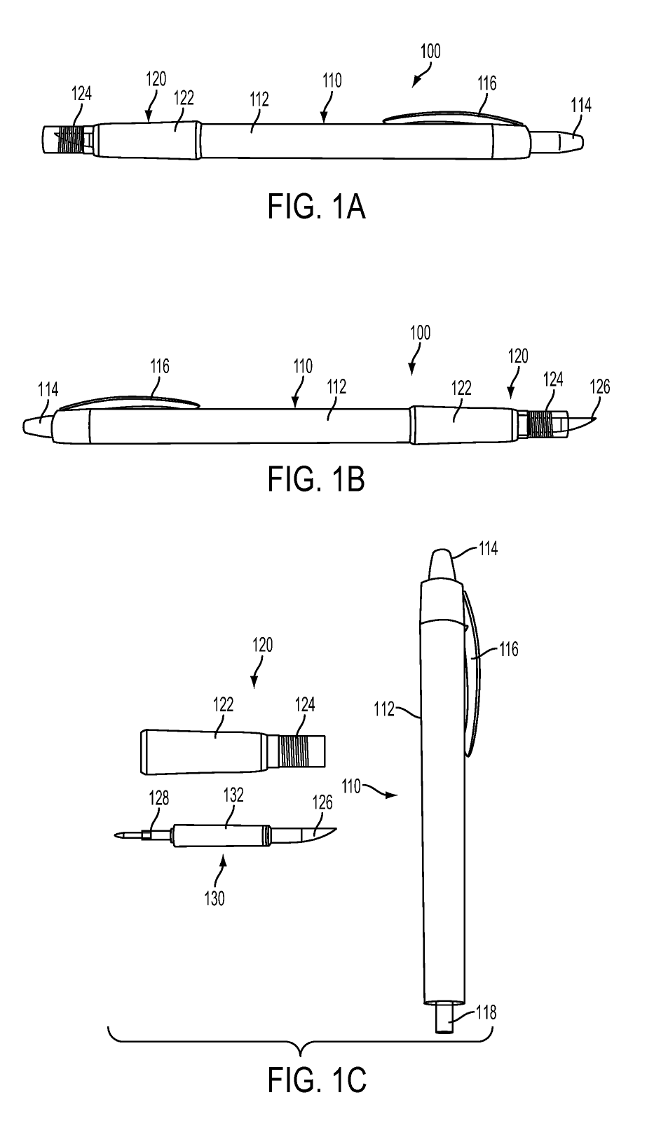

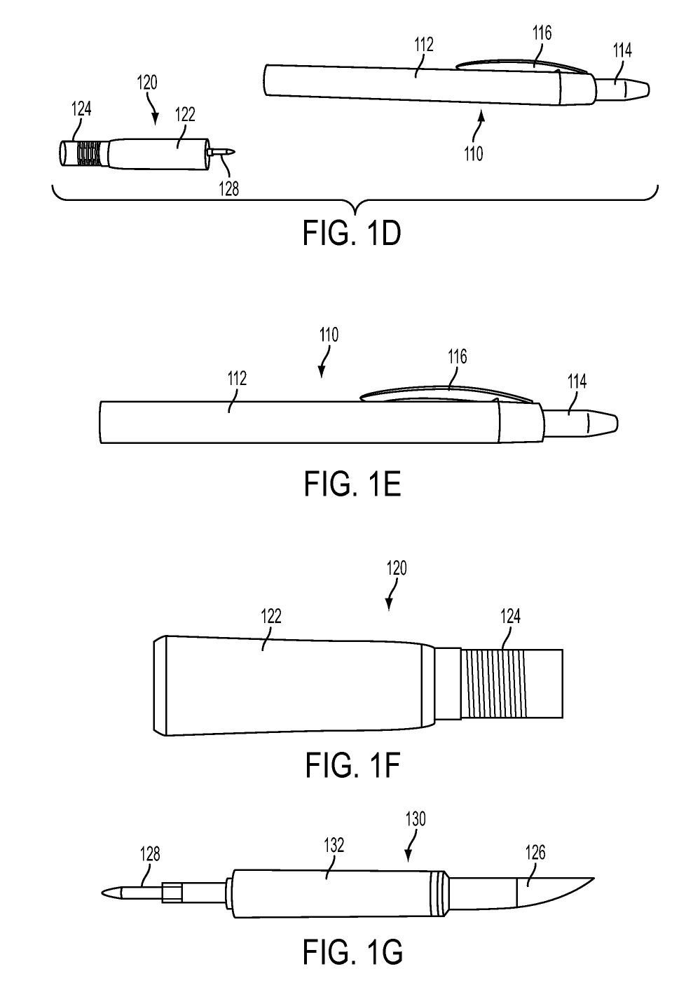

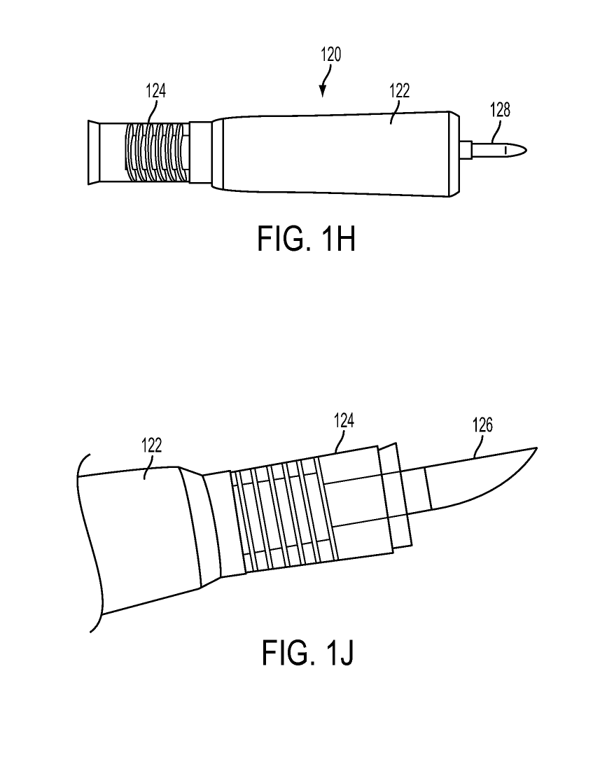

FIG. 1A illustrates an embodiment of an assembled prototype surgical scalpel assembly 100 in a retracted position. The cutting knife assembly 100 may include a handle assembly 110 and a blade tip assembly 120 in an assembled configuration. The visible portions of the handle assembly 110 may include a hollow barrel 112 and an activation button 114 (shown in a “retracted” position, wherein it extends further out of the hollow barrel 112 so as to allow an internal plunger to “retract”). The visible portions of the blade tip assembly 120 include a sheath 124, which has a grip portion 122 to aid in handling. In this embodiment, the handle assembly 110 may resemble major portions of a retractable pen and operate in a similar fashion. The button 114 may operate a cam follower (not illustrated) relative to a cam (not illustrated) so as to move a plunger (similar to a pen refill) in a known manner between retracted and extended positions. Various types of cam/cam follower arrangements are known in the art and are suitable for use in the various embodiments. For example, details of cam/cam follower mechanisms are disclosed in U.S. Pat. Nos. 3,819,282, 2,905,147, and 2,622,561, which are hereby incorporated by reference for purposes of such disclosures. Therefore, for purposes of simplicity, the cam/cam follower mechanism will not be described in further detail.

A spring (not illustrated in FIG. 1A) may be mounted between the barrel 112 and the plunger (also not illustrated in FIG. 1A) to compress during plunger extension and to expand during plunger contraction, as typically occurs during operation of a retractable pen. The handle assembly 110, which employs this type of extension and retraction, can be referred to as a pen-type handle. Although shown as having a clip 116 and resembling a pen with a round cross-section, embodiments of the handle assembly 100 are not so limited and may employ any cross-section, regular or irregular, and surface that is currently used or would be suitable for desired handling of a surgical instrument.

FIG. 1B illustrates the embodiment of the cutting knife assembly 100 from FIG. 1A in an extended position. The activation button 114 is shown in an “extended” position wherein less of the button is visible because it extends further into the barrel 112 so as to “extend” an internal plunger. As explained further below, the plunger engages and moves the cutting blade 126 to a position such that it extends from the sheath 124. In the illustrated embodiment, the cutting blade 126 is a surgical scalpel blade. The operation of the step down positions of the internal cam/cam follower fix the cutting blade in this visible, extended position for use until such time as the button 114 is operated again and the operation of the step down positions of the internal cam/cam follower act to retract the cutting blade 126 and return to the position illustrated in FIG. 1A.

FIGS. 1C, 1E, 1F, and 1G illustrate the embodiment of the cutting knife assembly 100 from FIG. 1A in a disassembled state, with the cutting knife assembly 130 comprising a cutting blade 126 removed from the sheath 124. The cutting blade 126 is mounted to a bearing surface 132 that is further mounted to a proximal end 128. In an exemplary embodiment, the bearing surface 132 is mounted to a spring (not illustrated) inside sheath that biases the cutting knife assembly in a retracted position within the sheath 124 for safety purposes. The proximal end 128 may be dimensioned and configured to engage the plunger 118 of the handle assembly 110, such as by a friction fit or other known means. As shown in FIG. 1C, the button 114 is pushed in and the operation of the step down positions of the internal cam/cam follower act to extend the plunger 118 to a position that, if assembled, would engage the proximal end 128 and cause the cutting blade 126 to extend out of the sheath 124 for use. As shown in FIG. 1E, the button 114 is released and the operation of the step down positions of the internal cam/cam follower act to retract plunger 118 (not shown) to a position that, if assembled, would cause the cutting blade 126 to retract into the sheath 124 for safe storage.

FIGS. 1D, 1E, and 1H illustrate an embodiment of the cutting knife assembly 100 from FIG. 1A in a disassembled state, with the cutting blade 126 largely hidden from view in its stored position within the sheath 124 such that only the proximal end 128 of the cutting blade (which lacks a sharp cutting surface) extends from the proximal end of the sheath 124 adjacent the grip portion 122.

FIG. 1J illustrates a close up view of a distal end of the embodiment of the cutting knife assembly 100 from FIG. 1B with the cutting blade 126 in an extended position. The cutting portion of the cutting blade 126 is illustrated as having a curved edge, as may be suitable for a surgical scalpel blade, but other types of blades may be use as discussed above including any known or desired cutting edge. The distal opening in the sheath 124 is illustrated as having a circular opening with a flared edge, but the sheath 124 may take any form that allows the cutting portion of the cutting blade 126 to move between stored and extended positions and provide desired functions. The flared opening illustrated in FIG. 1B may assist in the collection of contaminated material within the sheath 124 for disposal. In other applications, a smaller slit opening may be desirable to allow more clearance adjacent the cutting portion of the cutting blade 126. In general, the opening size and standoff distance of the cutting portion of cutting blade 126 when stored should be sufficient to prevent a finger or similar body part from contacting the sharp portion of the cutting blade 126.

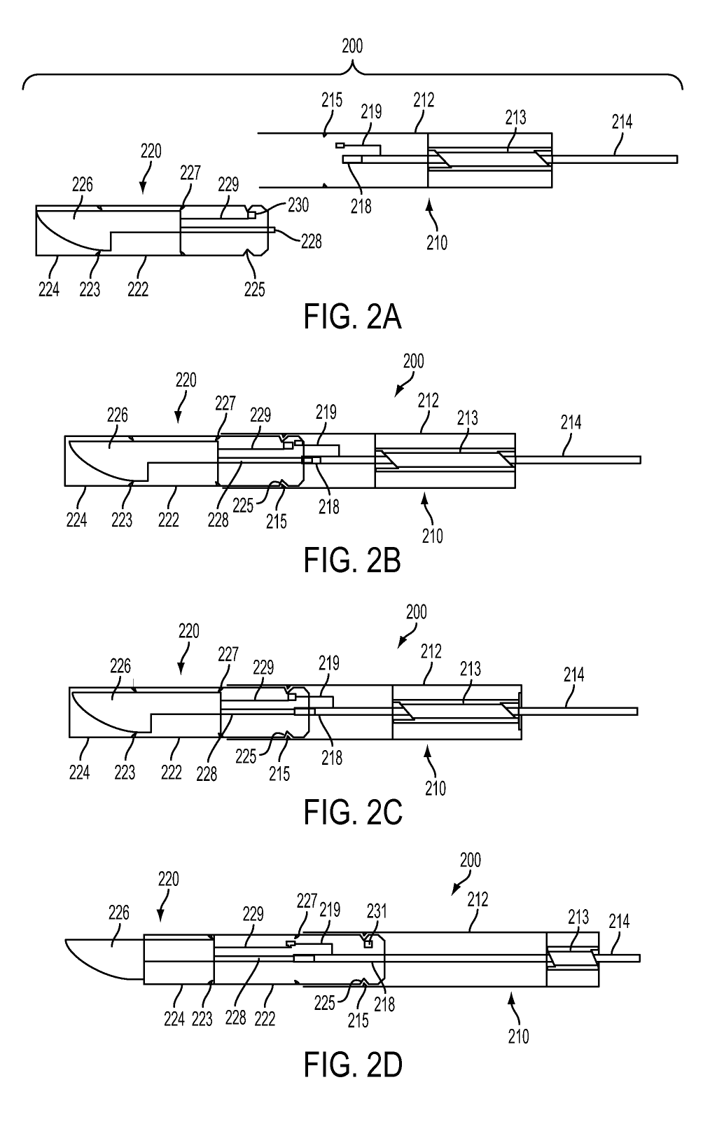

FIG. 2A shows a disassembled blade tip assembly 220 and handle assembly 210 in accordance with another embodiment. FIG. 2A, as well as FIGS. 2B-2D are schematic because certain details, such as the operation of spring 213, the operation of button 214 through a cam follower on plunger 218, and the operation of release mechanism 219 on lock elements 229/231, are illustrated conceptually rather than in realistic detail. Thus, while the figures illustrate that the spring 213 is compressed, and element 219 releases element 229 from element 231 in FIGS. 2C and 2D, the illustrated spring location and element deflection are for illustration purposes only and not intended to limit the scope of the claims.

In an embodiment the blade tip assembly 220 may include a sheath 224 that has a grip portion 222 and encloses a cutting blade 226. The cutting blade 226 may include a proximal end 228 positioned and dimensioned to engage the plunger 218 of the handle assembly 210. The interior of sheath 224 may include guides/stops 223/227 to control the position of the cutting blade 226. The cutting blade 226 may include a resilient locking member 229 that may include a catch portion that engages a recess or similar corresponding structure 231 on the interior of the sheath 224. In this manner, the cutting blade 226 can be locked in a safe position inside the sheath 224.

In this embodiment, the proximal end of the sheath 224 adjacent the grip portion 222 may be dimensioned slightly smaller than the distal opening in the hollow barrel 212 of the handle assembly 210 so that it may be inserted into the barrel 212 to attach the blade tip assembly 220 to the handle assembly 210. The proximal end of the sheath 224 may further include a structure (e.g., an indentation) 225 for securing to a corresponding structure (e.g., a protrusion) 215 inside the hollow barrel 212. This attachment is merely exemplary, and it is possible to use other forms, such as a blade tip assembly 220 that slides over the handle assembly 210, or a blade tip assembly 220 that abuts or is joined by flanges to the handle assembly 210. In other embodiments, other mechanisms may be used, such as bayonet mounts, screw threads, keyed friction fits, and the like to removably secure the blade tip assembly 220 to the handle assembly 210. In cases where the resilient locking member 229 must align with a corresponding release member 219, the attachment mechanism may use keyed elements or surfaces to assure the proper orientation of the members 229 and 219. Additionally, the handle assembly may use guides for the plunger 218 to assure proper orientation of the release member 219, in much the same way the guides 223/227 control orientation of the resilient locking member 229.

FIG. 2B shows an assembled interchangeable surgical scalpel 200 including a blade tip assembly 220 attached to the handle assembly 210 in accordance with the embodiment illustrated in FIG. 2A. The interchangeable surgical scalpel 200 is shown prior to engagement of the button 214, with the spring 213 in an extended position (not yet maximally compressed). The plunger 218 only partially engages the proximal end 228 of the cutting blade 226, and the release member 219 does not yet contact the resilient locking member 229.

In FIG. 2C, the button 214 is starting to be pushed. The plunger 218 fully engages the proximal end 228 of the cutting blade 226, and the release member 219 contacts the resilient locking member 229 to release it from the corresponding structure 231 on the interior of the sheath 224.

In FIG. 2D, the button 214 has been pushed and released, such that the operation of the step down positions of the internal cam/cam follower fix the cutting blade 226 in the extended position for use until such time as the button 214 is operated again and the operation of the step down positions of the internal cam/cam follower act to retract the cutting blade 226 and return to the positions illustrated in FIGS. 2C and 2B, so as to retract the cutting blade 226 into the sheath 224 and engage the resilient locking member 229 back into the corresponding structure 231 so as to secure the cutting blade 226 safely in the sheath 224. When use is complete, the blade tip assembly 220 can be removed, as illustrated in FIG. 2A, and disposed of or reused (such as through sterilization via an autoclave). The handle assembly 210 may be made of materials that may be sterilized in an autoclave such that the handle assembly 210 may be reused.

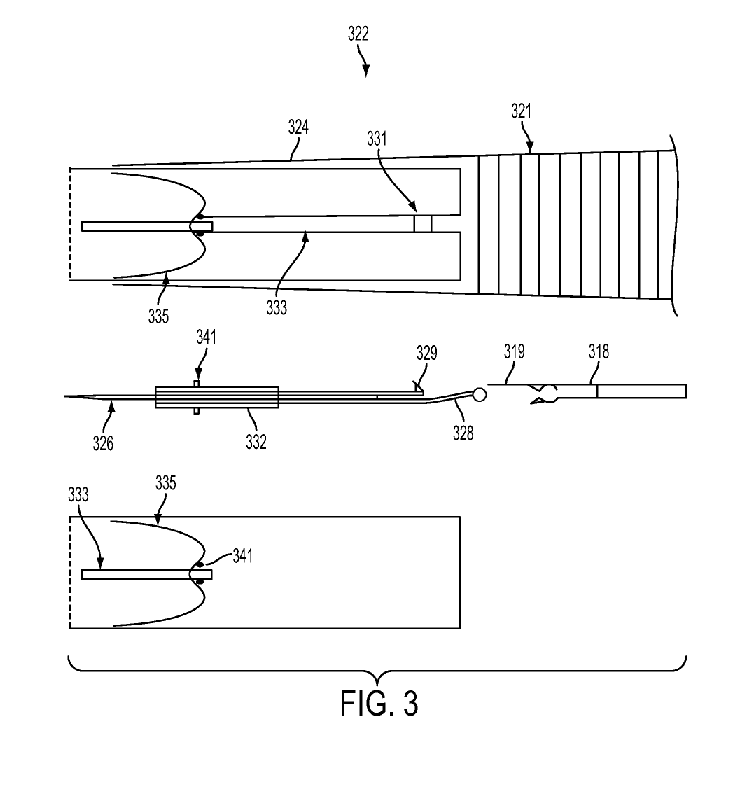

FIG. 3 is a cutaway/exploded view of interchangeable blade tip 322 components in accordance with yet another embodiment. A proximal end of the sheath 324 of the blade tip assembly 322 may include a ribbed grip 321 to aid in handling of the blade tip assembly 322 and the assembled interchangeable surgical scalpel. The sheath 324 may include guides/channels 333 to control the orientation of the cutting blade 326. A pin 341 may be positioned on the cutting blade 326 to engage a bar spring 335 mounted between the cutting blade 326 and the sheath 324. In this embodiment, the bar spring 335 acts to retract the cutting blade 326 into the sheath 324 when at rest.

The proximal end 328 may include a protrusion for initial engagement in a corresponding portion of the plunger 318. The plunger 318 has a further portion and an end wall for full engagement with the proximal end 328. During full engagement, a key or release member 319 may engage an angled surface of the resilient locking member 329 so as to release it from the corresponding structure 331 on the inside of the sheath 324. The plunger 318 can then act to move the cutting blade 326 along the guide/channel 333 and out of the sheath 324 against the forces of the bar spring 335.

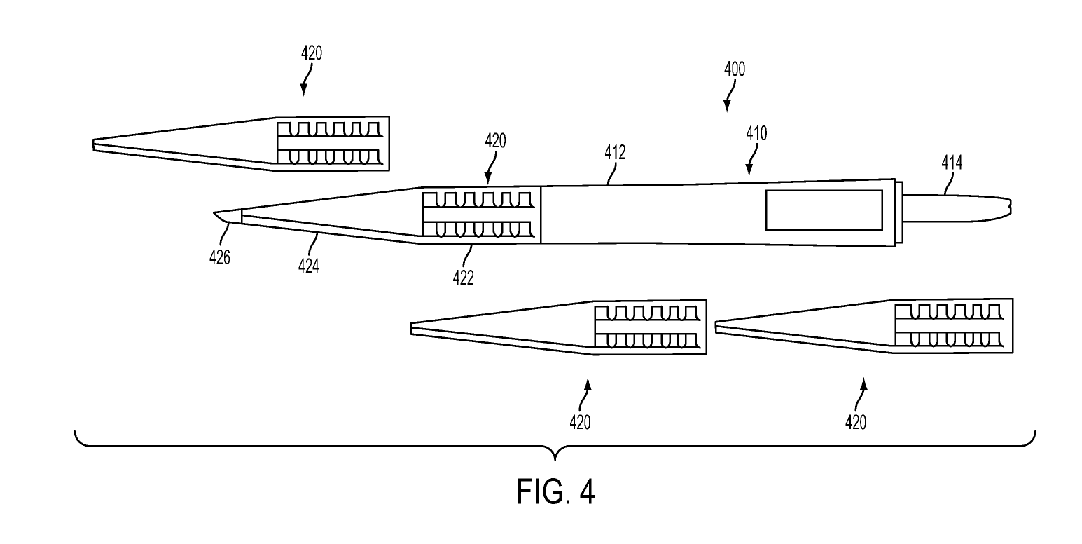

FIG. 4 is shows an interchangeable cutting knife 400 and a plurality of interchangeable blade tips 420 in accordance with a further embodiment. In use, multiple blade tips 420, which may have the same or different shaped cutting surfaces, can be used interchangeably with the same handle assembly 410, which may be made of autoclavable materials so that it may be reusable in medical applications. In an embodiment suitable for surgical applications, multiple scalpels may be used with only the need to autoclave a single handle assembly 410. This may reduce costs since disposable tips are cheaper than full scalpels, and different scalpels can be used with only one handle. This may improve safety since cutting blades are locked inside the blade tip assemblies until handed to a surgeon and remain locked and safe at all other times. The ease of operation and the positive engagement of a pen-style “click” may also avoid accidental injuries.

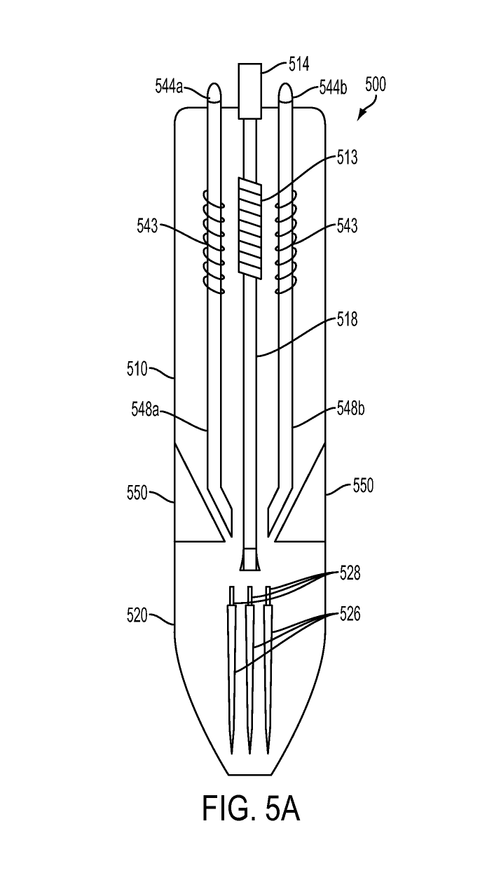

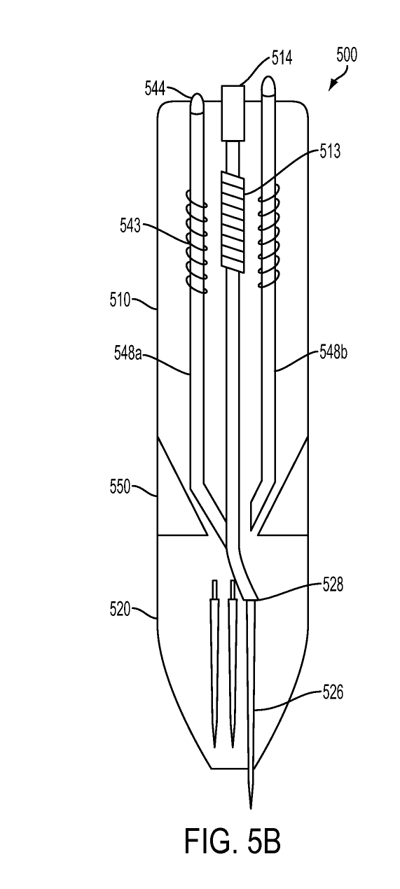

FIG. 5A and 5B show an embodiment of a surgical scalpel 500. FIGS. 5A-5B are schematics because certain details, such as the operation of spring 513, the operation of button 514 through a cam follower on plunger 518, and the operation of blade switching elements are illustrated conceptually rather than in realistic detail. Thus, while the figures illustrate that the spring 513 is compressed, and elements 544, 543, 548, and 550 can be used to selectively deflect plunger 518, the illustrated spring locations and element deflections are for illustration purposes only and not intended to limit the scope of the claims.

The embodiment interchangeable surgical scalpel 500 illustrated in FIG. 5A and 5B may incorporate multiple cutting blades 526 into a single blade tip assembly 520. The handle assembly 510 includes a blade-deployment button 514 for activating a blade-deployment plunger 518, which is shown in a retracted position in FIG. 5A. The handle assembly 510 can further include one or more alternate-scalpel selection buttons 544a, 544b. These alternate-scalpel selection buttons 544a, 544b act in concert with springs 543 and a cam follower mechanism (not illustrated) to move alternate-scalpel selection plungers 548a, 548b into a deployed or retracted position. In the deployed position, each alternate plunger 548a, 548b may deflect off of ramps or wedge elements 550 so as to positioned to deflect the blade-deployment plunger 518 so that it contacts different ones of the alternate cutting blades 526, as illustrated in FIG. 5B.

In FIG. 5B, one alternate button 544a is shown depressed which deploys the corresponding alternate-scalpel selection plunger 548a causing it to be deflected by the wedge element 550 to interact with the blade deployment plunger 518 so that when the blade-deployment button 514 is depressed, the blade deployment plunger 518 is deflected by the alternate-scalpel selection plunger 548a so as to engage the proximal end 528 of an alternate blade 526 and move it along guides (not illustrated) to an extended position. In the illustrated embodiment, the end of the blade deployment plunger 518 is flared so as to engage the proximal ends 528 of the scalpels at various deflection angles. However, other structures may be used for engaging the proximal ends 528 of the scalpels, such as plunger guides (not illustrated) and an interface positioned between the scalpels and the end of the blade deployment plunger 518. Additionally, structures for aligning the handle assembly 510 and the blade tip assembly 520 are not illustrated in the schematic views for simplicity. However, as with the embodiment of FIGS. 2A-2D, numerous known mechanisms may be used for registration and alignment of the plungers and other moving structures.

While this multiple-blade embodiment is disclosed as using flexible plungers 518 and 548 to alternately select particular surgical cutting blades 526, other mechanisms such as typically used in multi-color pen or pencil mechanisms may be used. In such embodiments, each separate plunger activates a separate cutting blade. Such alternate selection mechanisms are disclosed in U.S. Pat. Nos. 2,113,320, 2,849,983, 3,260,242, 3,700,340, 3,989,389, and 6,899,482, which are hereby incorporated by reference.

While the various embodiments have been described above as surgical scalpels, the embodiments are not limited to medical applications. More generally, the embodiments are usable with any cutting knife that implements the structures for deploying and retracting blades described above that may be useful for a variety of applications, particularly applications in which a retractable blade provides important safety advantages. Thus, the claims are not limited to surgical scalpels unless specifically recited in the claims themselves.

The preceding description of the embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the widest scope consistent with the following claims and the principles and novel features disclosed herein. Further, any reference to claim elements in the singular, for example, using the articles “a,” “an,” or “the” is not to be construed as limiting the element to the singular.

What is claimed is:

1. A cutting knife, comprising:
a pen-type handle comprising:
an activation button at a proximal end; and
a spring-loaded plunger, wherein the activation button is configured to move the spring-loaded plunger between retracted and extended positions at a distal end of the pen-type handle; and
an interchangeable blade tip comprising:
a sheath dimensioned to removably attach to a proximal end to the distal end of the pen-type handle; and
a spring-loaded cutting blade configured to engage the spring-loaded plunger and extend to an exposed use position when the spring-loaded plunger is in the extended position, and further configured to disengage the spring-loaded plunger and retract to a safe position when the spring-loaded plunger is in the retracted position.

2. The cutting knife of claim 1, wherein:
the spring-loaded cutting blade further comprises a resilient member that engages a portion of the sheath when in a retracted position so as to lock the cutting blade within the sheath; and
the spring-loaded plunger comprises a member that disengages the resilient member of the cutting blade from the sheath during initial movement into an extended position so as to unlock the cutting blade from the sheath.

3. The cutting knife of claim 2, wherein:
the resilient member comprises a tapered catch at an end of a leaf spring.

4. The cutting knife of claim 2, wherein the sheath and pen-type handle are dimensioned to align the resilient member of the cutting blade and the member that disengages on the plunger.

5. The cutting knife of claim 1, wherein the pen-type handle is made from autoclavable materials.

6. The cutting knife of claim 1, wherein the interchangeable blade tip is made from disposable materials.

7. The cutting knife of claim 1, wherein the interchangeable blade tip further comprises a grip surface on a proximal portion of the sheath.

8. The cutting knife of claim 1, wherein the interchangeable blade tip further comprises a channel on an inner portion of the sheath to guide the cutting blade.

9. The cutting knife of claim 1, wherein the interchangeable blade tip further comprises a bar spring between an inner portion of the sheath and the cutting blade.

10. The cutting knife of claim 1, wherein:
the interchangeable blade tip further comprises a plurality of cutting blades; and
the pen-type handle comprises:
a plurality of activation buttons at the proximal end; and
a plurality of spring-loaded plungers associated with each of the plurality of activation buttons, wherein each of the plurality of activation buttons is configured to move a spring-loaded plunger between retracted and extended positions at a distal end of the pen-type handle.

11. The cutting knife of claim 10, wherein the plurality of spring-loaded plungers comprise:
a blade-deployment plunger, wherein a blade-deployment button among the plurality of activation buttons is configured to move the blade-deployment plunger between retracted and extended positions at a distal end of the pen-type handle to engage a proximal end of one of the plurality of cutting blades; and
at least one alternate-scalpel selection plunger, wherein at least one alternate-scalpel selection button among the plurality of activation buttons is configured to move the at least one alternate-scalpel selection plunger between a retracted position and an extended position that acts against a wedge element so as to deflect the blade-deployment plunger to engage an alternate one of the plurality of cutting blades.

12. The cutting knife of claim 1, wherein the cutting blade is a surgical scalpel blade.

13. A surgical scalpel, comprising:
a handle comprising:
a hollow barrel having an inner surface, a proximal opening in a proximal end and a distal opening in a distal end;
a button having proximal end, a distal end, and a flange, wherein the button is dimensioned to extend its proximal end through the proximal opening of the hollow barrel and be retained by the flange;
a cam on the inner surface of the hollow barrel;
a cam follower positioned adjacent the distal end of the button and configured to engage the cam on the inner surface of the hollow barrel to move between an extended position and a retracted position;
a plunger having a proximal end, a distal end, and spring stop, wherein the proximal end of the plunger is dimensioned to engage a distal end of the cam follower; and
a plunger spring configured to engage the spring stop on the plunger and the hollow barrel so as retract the plunger from an engagement position when the cam follower is in the retracted position; and
an interchangeable blade tip comprising:
a hollow sheath having a proximal opening in a proximal end, a distal opening in a distal end, blade guides on an inner surface, and a blade stop at a distal end of the blade guides, wherein the proximal end is dimensioned for removable attachment to the distal opening of the hollow barrel;
a cutting blade having a cutting surface at a distal end, a longitudinal edge for engaging the blade guides, a spring stop, and a proximal end dimensioned to engage a distal end of the plunger when the plunger is in the engagement position; and
a blade spring configured to engage the spring stop on the cutting blade and the hollow sheath so as retract the cutting blade within the sheath when the plunger is disengaged from the proximal end of the cutting blade.

14. The cutting knife of claim 13, wherein:
the cutting blade further comprises a resilient member that engages a portion of the hollow sheath when in a retracted position so as to lock the cutting blade within the hollow sheath; and
the plunger further comprises a member that disengages the resilient member of the cutting blade from the hollow sheath during initial movement into an extended position so as to unlock the cutting blade from the hollow sheath.

15. The cutting knife of claim 14, wherein:
the resilient member comprises a tapered catch at an end of a leaf spring.

16. The cutting knife of claim 14, wherein the proximal end of the hollow sheath and distal end of the hollow barrel are dimensioned to align the resilient member of the cutting blade and the member that disengages on the plunger.

17. The cutting knife of claim 13, wherein the handle is made from autoclavable materials.

18. The cutting knife of claim 13, wherein the interchangeable blade tip is made from disposable materials.

19. The cutting knife of claim 13, wherein the interchangeable blade tip further comprises a grip surface on a proximal portion of the sheath.

20. The cutting knife of claim 13, wherein the interchangeable blade tip further comprises a plurality of cutting blades; and
the handle comprises:
a plurality of activation buttons at the proximal end;
a plurality of cam followers engaging the plurality of activation buttons; and
a plurality spring-loaded plungers associated with each of the plurality of activation buttons, wherein each of the plurality of activation buttons and cam followers is configured to move a spring-loaded plunger between retracted and extended positions at a distal end of the handle.

21. The cutting knife of claim 20, wherein the plurality of spring-loaded plungers comprise:
a blade-deployment plunger, wherein a blade-deployment button among the plurality of activation buttons is configured to move the blade-deployment plunger between retracted and extended positions at a distal end of the pen-type handle to engage a proximal end of one of the plurality of cutting blades; and
at least one alternate-scalpel selection plunger, wherein at least one alternate-scalpel selection button among the plurality of activation buttons is configured to move the at least one alternate-scalpel selection plunger between a retracted position and an extended position that acts against a wedge element so as to deflect the blade-deployment plunger to engage an alternate one of the plurality of cutting blades.

22. The cutting knife of claim 13, wherein the cutting blade is a surgical scalpel blade.
